(19) United States
(12) Patent Application Publication
(10) Pub. No.: US 2003/0149259 A1
(43) Pub. Date: Aug. 7, 2003

(54) Foot and mouth disease virus diagnostic and methods

(76) Inventors: Johnny Dale Callahan, Severn, MD (US); William Max Nelson, Potomac, MD (US); Beverly L. Mangold, Rockville, MD (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/147,920

(22) Filed: May 20, 2002

Related U.S. Application Data
(60) Provisional application No. 60/291,636, filed on May 18, 2001.

(51) Int. Cl. C12Q 1/70; C12Q 1/68; C12P 21/08; G01N 33/53; C07H 21/04; A61K 39/125; C12N 7/00; C07K 16/00

(52) U.S. Cl. 536/24.32; 435/5; 435/6; 435/7.1; 536/23.72; 435/235.1; 530/388.3; 424/216.1

(57) ABSTRACT

The invention relates to diagnostic methods, probes, detection systems and kits for the identification of foot and mouth disease virus (FMDV) infection in a biological sample obtained from a farm animal. It was discovered that a highly conserved region of sequence existed with the 3D coding region of the FMDV genome. This region was found to be strikingly similar, and often identical or with only one or two nucleotide substitution, between the various serotypes of FMDV. Thus, by performing PCR analysis with probes comprising sequences form this region or ELISA with antibodies directed to polypeptide products expressed from this region, a plurality of serotypes of FMDV could be detected from a single test. Further, by including dried PCR reagents plus trehelose, kits could be stored at room temperatures for long periods of time without any significant loss in sensitivity or specificity. Thus, FMDV assays could be performed on site, within the field and quickly so that a diagnosis of FMDV infection can be made within hours.

### REFERENCE TO RELATED APPLICATIONS

The present application claims priority to U.S. Provisional Application Serial No. 60/291,636 entitled “Rapid and Specific Foot and Mouth Disease Virus TaqMan Assay using a Portable Instrument and Dried RT-PCR Reagents,” filed May 18, 2001, the contents of which are hereby entirely and specifically incorporated by reference.

### BACKGROUND

1. Field of the Invention

The present invention relates generally to diagnostic methods and probes, detection systems and kits for identifying foot-and-mouth disease virus (FMDV), and in particular, to systems and kits that are capable of detecting multiple serotypes of the foot and mouth disease virus.

2. Description of the Background

Events in the United Kingdom have demonstrated very clearly that foot-and-mouth disease virus (FMDV) is so highly contagious that rapid diagnosis is required to control its spread. See, i.e., Adam, D.: Nature 410, 398 (2001) and Enserink, M: Science 291, 2298-2300 (2001). At present, routine diagnosis of FMDV can be made at the OIE/FAO World Reference Laboratory for FMD at Pirbright, Surrey, UK, by the combined use of serological and virus isolation techniques, supplemented by the reverse transcriptase polymerase chain reaction (RT-PCR). These techniques require the availability of a dedicated laboratory facility. A recent report described the use of a TaqMan RT-PCR for detection of FMDV type O in pathogenic studies of FMDV in pigs: See, i.e., Alexanderson, S, et al.: J Gen Virol 82(4), 747-55 (2001) and J Gen Virol 2001 April;82(Pt 4):747-55.

Foot-and-Mouth Disease Virus (FMDV) is actually a group of closely related viruses, members of the genus aphthovirus and family Picomaviridae. The genus aphthovirus has two members, FMDV and Equine Rhinitis A Virus (ERV-1). The second genus member, ERV-1, shares some sequence homology with FMDV, but is not a cause of FMD. ERV-1 is the agent of an equine respiratory disease (horses are not suceptable to FMDV). The seven serotypes of FMDV include types A, O, C, Asia 1, Sat 1, Sat 2, and Sat 3. Serotypes are distinguishable by serotype specific enzyme linked immunosorbent assays (ELISA).

Because of the range of species affected, the high rate of infectivity, and the fact that FMDV is shed before clinical signs occur, FMD is one of the most feared reportable disease in North America. Disease caused by FMDV is devastating to farm animals and can have a major economic impact on countries producing cloven-hoved animals (cattle, pigs, sheep, goats and camelids) or their products. An outbreak of FMD would, (and has in the past) cost millions of dollars in lost production, loss of export markets, and loss of animals during eradication of the disease. The significance of several other reportable vesicular diseases is due primarily to their close resemblance to FMD which makes distinguishing between them a high priority at the earliest indications of an unusual disease outbreak.

In the last 100 years, six FMD epizootics have occurred in the United States: one each in 1902, 1908, and 1914; two in 1924; and the last in 1929. Of these, the largest and most severe outbreak began in 1914 (see, i.e., Damiant, G., January 1., 172(1):45-54 (1978)). The primary control method utilized in the past by the Bureau of Animal Husbandry and current policy of the Agricultural Research Service (USDA) for each FMD epizootic is described as the “stamping-out” method of eradication. This consisted of inspection, quarantine, slaughter, and disposal of infected and exposed animals, and subsequent testing of properties in and around outbreak areas with susceptible animals. In the six epizootics from 1902 to 1929, more than 324,000 head of livestock were slaughtered. Direct costs and indirect losses were estimated at $253 million. The United States has remained free of foot-and-mouth disease since 1929; however, FM is still a major problem worldwide and is a major constraint to the trade of live animals and their products. The combined loss of animals due to eradication efforts and the subsequent loss of access to world markets lead to enormous economic consequences. A recent example was Taiwan (1997), where an outbreak in pigs cost the government hundreds of millions of dollars.

In FMD epizootics, large amounts of virus are excreted by infected animals before clinical signs are evident. The disease is highly contagious and may be spread over long distances by winds and by the movement of infected or contaminated animals, products, objects, or people. FMD has a low mortality rate in adult animals, but often has a high mortality rate in young animals due to myocarditis.

The epizootiology of FMD is a complex and not fully understood interaction of viral strain, animal host, and environmental factors. Considerable research efforts have been directed towards the understanding of mechanisms of persistence and identification of the carrier animal, however, experiments to demonstrate transmission of FMDV from carriers to susceptible in-contact animals have been unsuccessful. The relative importance of known carrier animals (cattle, sheep, and buffalo) remains poorly understood. Virus can persist in dogs, cats, and other small animals. Wild game animals may also play an important role in the transmission cycle through their migratory habits which facilitate long-range movement of the virus.

FMD can be transported over great distances through infected animals and their products. The virus remains viable in frozen meats for up to three months and up to two months in ham, bacon, and certain sausages. The virus persists for longer periods in lymph nodes and bone marrow, and when discarded as garbage, such as from abittiors, can constitute a mechanism for infection for dogs, cats, and swine. Due to the economic and political significance of FMD and it's similarity to other vesicular diseases: vesicular stomatitis virus (VSV), swine vesicular disease (SVD), and vesicular exanthema of swine (VES), a rapid definitive diagnosis is essential.

A number of articles have been published describing RT-PCR methods for FMDV. See i.e. Reid et al. “Diagnosis of foot-and-mouth disease by real-time flurogenic PCR assay” The Veterinarv Record, pp. 621-623 Nov. 17, 2001; Nunez et al., “RT-PCR in foot-and-mouth disease diagnosis,” and Alexandersen et al., “The early pathogenesis of foot-and-mouth disease in pigs infected by contact: a quantitative time-course study using TaqMan RT-PCR, Journal of General Virology, 82, 747-755 (2001). The FMD assay described by Alexandersen is a probe hydrolysis based RT-PCR assay, and limited to the detection of a single serotype of the virus. In addition, U.S. Pat. No. 6,048,538 to Yi Wang et al., relates to peptides derived from the non-structural proteins of FMDV as diagnostic reagents, and targets non-structural proteins 3A, 3B and 3C.

### SUMMARY OF THE INVENTION

The present invention overcomes the problems and disadvantages associated with current strategies and designs and provides compositions and methods for the detection of foot and mouth disease virus.

One embodiment of the invention is directed to isolated nucleic acids comprising a sequence of any of at least ten contiguous nucleotides of a portion of the 3D coding region of a FMDV genome, wherein said portion comprises the 3′-terminal third of said coding region or of the complement of said portion; or that hybridizes under stringent hybridization conditions to said portion or the complement of said portion. Preferably, the nucleic acid sequence comprises at least twenty contiguous nucleotides, more preferably at least thirty contiguous nucleotides, even more preferably at least thirty five contiguous nucleotides, and, in certain embodiment, still more preferably at least fifty contiguous nucleotides. The nucleic acid may comprises DNA or PNA, which may be synthetically synthesized, or recombinantly produced from a vector or bacterial or eukaryotic cell containing the sequence. Preferably, the sequence is derived from a 3′-terminal portion of the 3D coding region of FMDV.

Another embodiment of the invention is directed to kits for the detection of a FMDV infection in a patient comprising a pair of nucleic acids of the conserved region of the FMDV genome for use in PCR amplification. Kits can detect a plurality of serotypes of FMDV, preferably all serotypes. Kits preferably contain dried reagents for RT-PCR analysis of the biological sample plus trehalose. Preferably, kits are portable and can be taken and used in the field. Detection can be performed within about two hours or less, or stored at room temperatures (i.e. about 20-24° C.), for at least a year or more.

Another embodiment of the invention is directed to methods for detecting a FMDV infection in a patient comprising amplifying a portion of nucleic acid of a biological sample obtained from the patient by PCR amplification to produce an amplification product wherein the amplification product contains a sequence derived from the 3D coding region of a FMDV genome; and detecting said FMDV infection in the patient by the presence of the amplification product. These methods detect the presence of an FMDV infection in patients such as farm animals, and can detect an infection caused by more than one of the serotypes such as serotypes Asia 1, A, C, O, Sat 1, Sat 2, and Sat 3. The methods preferably involves PCR RT-PCR, or probe hydrolysis RT-PCR amplification of the FMDV genome from the biological sample, which may be a sample of tissue, fluid or combination of tissue and fluid obtained from the patient. In addition, methods can distinguish an FMDV-infected patient from a patient infected with one or more of the viruses selected from the group consisting of swine vesicular disease virus, vesicular stomatitis virus, and vesicular exanthema of swine virus, or a FMDV-vaccinated patient from a FMDV-infected patient.

Another embodiment of the invention is directed to methods for detecting a FMDV infection in a patient comprising contacting a biological sample obtained from the patient with an antibody directed against a 3D coding region of an expressed portion of a FMDV genome to form antibody/antigen complexes; and detecting the FMDV infection in the sample by the presence of antibody/antigen complexes. Methods can detect a plurality of serotypes of FMDV including serotypes Asia 1, A, C, O, Sat 1, Sat 2, and Sat 3. Antibodies may be monoclonal, polyclonal, or fragments such as Fv fragments.

Another embodiment of the invention is directed to antibodies specific for products expressed from the 3D coding region of the FMDV genome and kits containing such antibodies. Preferably, antibodies are labeled with a detectable label to facilitate binding detection and the label is a fluorescent compound.

Other embodiments and advantages of the invention are set forth, in part, in the following description and, in part, may be obvious from this description, or may be learned from the practice of the invention.

### DESCRIPTION OF THE DRAWINGS

FIG. 1 Polypeptide structure of the FMDV genome.

### DESCRIPTION OF THE INVENTION

As embodied and broadly described herein, the present invention is directed to tools, methods and kits for the detection of FMDV. More specifically, the present invention is directed to nucleic acids that represent a conserved region of most and likely all serotypes of FMDV, and to probes, primers, and antibodies derived from this region as well as diagnostic kits and diagnostic method that contain these tools which can be used to detect FMDV in patients and the environment.

Conventional methods for the detection and identification of animals infected with FMDV are serotype specific. One set of reagents in a kit will only detect one specific serotype of FMDV. To identify more than one serotype requires an equal number of additional kits and testing. Further, these kits contain labile reagents so that assays must be performed in a clinical setting such as a laboratory. This adds both a considerable amount of time and expense to diagnostic procedures.

Seven distinct serotypes of FMDV have been identified to date, namely, serotypes O, A, C, Asia, Sat 1, Sat 2, and Sat 3, all identifiable by ELISA. Of these serotypes, there also exists variations in the different isolates. For example, serotype A includes isolates A10A, A10H, A12, A5, A24C, A24V, and A22; serotype O includes isolates O1C, O1K, O1L, and O2B; serotype C includes isolates C1G, C4TDF, C1H, C1O, C1SP, C1SDD, C2P, C3R, C5A, C3A, and C31; and serotype SAT2 includes isolates SAT2K and SAT2R. This list is by no means complete, but represents some of the more well known and better studied variations. Consequently, with this number of serotypes and ever increasing number of variations, it is very difficult and often impossible to make a diagnosis of FMDV infection (or absence of FMDV infection) without performing a test for each and every serotype and strain.

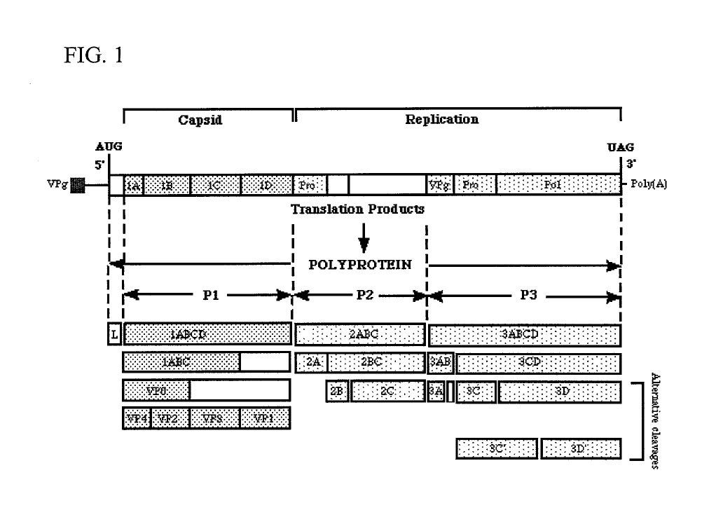

The FMDV genome (approximately 7-9 kb) consists of a single RNA positive strand encoding four structural proteins termed VP1, VP2, VP3, and VP4, and at least ten non-structural proteins (see FIG. 1). The non-structural proteins are encoded within sections of the genome referred to as P2 and P3. These sections can be further divided into regions 2A, 2B, and 2C, and 3A, 3B, 3C, and 3D, respectively. Various combinations of these regions encode proteins involved in viral replication. The principal viral replicase gene is located in the region known as 3D, which is about 1.5 kb is size.

It has surprisingly been discovered that a plurality of different serotypes of FMDV and likely all can be rapidly and accurately detected using kits and assays that can be performed both in the field and in a short amount of time. Kits are based on the discovery that there exists a highly conserved region of the FMDV genome, namely a region within the 3D region, to which reagents for diagnostic assays can be targeted. These reagents allow for the detection, identification and measurement of a plurality of serotypes of FMDV without requiring a user to perform multiple assays. The advantages in convenience and reduced expense, and the reduction in overall time to a diagnosis, are substantial. Further, this region is distinct from other, similar viral genome and the genomes of viruses that present similar pathological conditions and, thus, cause confusion in the field. These similar viruses include the aphthoviris such as equine rhinitis A virus (ERV-1), the erbovirus such as equine rhinitis B virus (ERV-2), the teschovirus virus such as porcine enterovirus 1, and the cardioviruses EMCV (B, D, R, and Mengo), and theilovirus (VHEV). Further, methods of the invention can distinguish between infected and uninfected, but vaccinated animals.

Description of Conserved Region of RNA Polymerase 3D

The conserved region of the FMDV genome can be found within the RNA polymerase gene which comprises approximately 1,500 base pairs. An area that has been identified as highly conserved within the RNA polymerase gene is referred to as 3D.

One embodiment of the invention is directed to isolated nucleic acids comprising a conserved sequence of the 3D coding region of a FMDV genome. The conserved portion is that portion or its complement which hybridizes under stringent hybridization conditions to similarly positioned sequences of the 3D regions of more than one (e.g. 3, 4, 5, 6, 7) different serotype of FMDV genome. More preferably, the conserved region hybridizes to the genome sequences of the conserved 3D regions of more than one serotype of FMDV. Preferably, the conserved nucleic acid of the invention comprises at least ten (e.g. 12, 14, 16, 18) contiguous nucleotides, at least twenty (e.g. 22, 24, 26, 28) contiguous nucleotides, at least thirty (e.g. 31, 32, 33, 34) contiguous nucleotides, at least thirty-five (e.g. 36, 38, 40, 42) contiguous nucleotides, and, in certain embodiments, at least fifty (e.g. 52, 54, 56, 58) contiguous nucleotides.

Table 1 illustrate some of the various probes, primers (forward and reverse) that can be used.

TABLE 1

Foot-and-Mouth Disease Virus (FMDV) Primers and Probes*
Reference Strain>gb|AF189l57

| Reference ID | Primer or Probe Sequence | Mer |
|---|---|---|
| 3D Gene | | |
| FMD-6770-F | 5′-CTg ggT TTT ATA AAC CTg TgA Tg-3′ | 23 (SEQ ID NO 1) |
| FMDV-6769-F | 5′-ACT ggg TTT TAC AAA CCT gTg A-3′ | 22 (SEQ ID NO 2) |
| FMD-6770-F-Hy | 5′-CTg ggT TTT ATA AAC CTg TgA T-3′ | 22 (SEQ ID NO 3) |
| FMDV-6769-F-Hy | 5′-ACT ggg TTT TAC AAA CCT g-3′ | 19 (SEQ ID NO 4) |
| FMD-6820-T1 | 5′-TCC TTT gCa CgC CgT ggg AC-3′ | 20 (SEQ ID NO 5) |
| FMD-6822-T2 | 5′-CTT TgC ACg CCg Tgg gaC CAT-3′ | 21 (SEQ ID NO 6) |
| FMDV-6874-R | 5′-gCg AgT CCT gCC ACg gA-3′ | 17 (SEQ ID NO 7) |
| FMDV-6872-R-Hy | 5′-AgT CCT gCC ACg gA-3′ | 14 (SEQ ID NO 8) |
| Second Assay | | |
| Name | Sequence | mer |
| FMD-7493-F1 | 5′-TCC gTg gCA ggA CTC gC-3′ | 17 (SEQ ID NO 9) |
| FMD-7494-F2 | 5′-CCg Tgg CAg gAC TCg C-3′ | 16 (SEQ ID NO 10) |
| FMD-7617-R1 | 5′-CAC ACg gCg TTC ACC CA-3′ | 17 (SEQ ID NO 11) |
| FMD-7616-R2 | 5′-aCa Cgg CgT TCA C-3′ | 13 (SEQ ID NO 12) |
| Probes | | |
| FMDV-7579-T2 | 5′-CTA CAg ATC ACT TTA CCT gCg-3′ | 21 (SEQ ID NO 13) |
| FMDV-7581-T1 | 5′-AgC TAC AgA TCA CTT TAC CTg-3′ | 21 (SEQ lID NO 14) |

* = “G” residues are shown in lower case simply to distinguish them clearly from “C” residues.

One or more of the residues of the conserved region can be modified, deleted or substituted without departing from the accuracy or ability of the present methods and kits to detect the various serotypes of FMDV. That is, a primer or probe can contain a one, two or three nucleotide substitution, and a set of nucleotides can change location by one or more base pairs and not affect the accuracy of the method of the inevntion. The invention includes sequences of the conserved region, sequences that are complementary to the conserved region, and also sequences that hybridize under stringent hybridization condition (e.g. see “Current Protocols in Molecular Biology” published by Wiley Interscience, 1998).

In addition, over the 300-312 base pair area of the highly conserved area in the 3D gene, there is a high degree of overlap among the particular target area within gene 3D. There are subsections within the approximately 300-312 residue area that can be used as targets, for example, 105 residue sections or 103 residue sections, all of which are very highly conserved and among which there is a very high degree of overlap between the various serotypes and isolates of FMDV, but no substantial overlap to any large degree with other related viruses that are not FMDV. The invention further comprises analogs, homologs, recombinant and synthetic versions of the sequences of the conserved region, as well primers or probes that hybridize to the primers and probes so identified.

In certain embodiments, the conserved region of the genome is located between approximately positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype, of the FVDM genome (see Table 2). The sequences of these regions, all of which are publicly available (for example in GenBank), correspond well with the sequences in the same regions for all isolates of FMDV.

TABLE 2
FMDV Sequences Embraced by Primers and Recognized by Probe

| | Residues | | | |
|---|---|---|---|---|
| Accession | Forward | Reverse | Sequence recognized | |
| No. | Primer | Primer | Probe | Gene/Genome |
| 1. AF207520 | 1186 | 1292 | 1237-1256 | Asia 1, polyprotein gene |
| 2. AF377945 | 7484 | 7590 | 7535-7554 | O/SKR/2000 |
| 3. AF308157 | 7800 | 7906 | 7851/7870 | Type O, Complete genome |
| 4. AF274010 | 7795 | 7901 | 7846/7865 | Type C, strain c-s8 |
| 5. AF026168 | 7403 | 7509 | 7454-7473 | Type O, Chu-Pei Strain |
| 6. AJ320488 | 7853 | 7959 | 7904-7923 | Type O, 01 Campos |
| 7. AF189157 | 6769 | 6875 | 6820-6839 | Type O, Strain 01 polyprotein |
| | | | | gene |
| 8. AF154271 | 7420 | 7526 | 7471-7490 | Type O, Strain - Tau- |
| | | | | YuanTW97, polyprotein |
| | | | | precursor |
| 9. AJ010871 | 80 | 186 | 131-150 | Type A, subtype A5 isolate |
| | | | | A5WW partial 3D gene and |
| | | | | 3′UTR |
| 10. AJ133359 | 7795 | 7901 | 7846-7865 | Strain C isolate rp146 |
| 11. AJ133358 | 7795 | 7901 | 7846-7865 | Strain C isolate rp99 |
| 12. AJ133357 | 7795 | 7901 | 7846-7865 | Strain C, isolate c-s8c1 |
| 13. V01136 | 1189 | 1295 | 1240-1259 | RNA Polymerase gene, |
| | | | | Serotype not specified |
| 14. X00871 | 7485 | 7591 | 7536-7555 | Type O, Strain 01K, gene: |
| | | | | polyprotein precursor |
| 15. X00429 | 6775 | 6881 | 6826-6845 | Type A, Strain A10-61, |
| | | | | polyprotein complement |
| 16. J02181 | 1189 | 1295 | 1240-1259 | RNA polymerase gene, |
| | | | | type not specified |
| 17. M11027 | 1365 | 1471 | 1416-1435 | type C-1 - Santa Pau (C-s8) |
| | | | | replicase (p61) gene, |
| | | | | complete eds, protein p18 |
| | | | | and 3′ extracistronic region |
| 18. M10975 | 7392 | 7498 | 7443-7462 | Virus A12; L, P2, and P3 |
| | | | | polypeptide coding region |
| 19. X74812 | 7487 | 7593 | 7538-7557 | Virus A L-fragment |
| 20. X85493 | 1186 | 1292 | 1237-1256 | Type A, subtye A22, 3D gene |
| 21. AJ251473 | 7455 | 7561 | 7506-7525 | SAT2 RNA L, VP4, VP2, |
| | | | | VP3, VP1, 2A, 2B, 2C, 3A, |
| | | | | VPg1, VPg3, pro coding |
| | | | | polypolyprotein |
| 22. AJ007572 | 7841 | 7947 | 7892-7911 | derived from C3Arg85, |
| | | | | clone 15 |
| 23. AJ007347 | 7841 | 7947 | 7892-7911 | polyprotein isolate |
| | | | | C3Arg85 |

One embodiment of the invention is directed to a method for detecting a plurality of serotypes of FMDV with a single assay. Detection is achieved by targeting a highly conserved region of the RNA genome, specifically the 3D region. The conserved sequence of about 300 nucleotides is within the coding region of the gene and ends approximately 100 nucleotides from the 3′ terminus of 3D. For example, in the Asia 1 serotype, this conserved region extends from positions 1,102 to 1,401 of the 3D gene. The region is very highly conserved between different serotypes, and also different strains and variants of FMDV, so much so that there exists less than ten, preferably less than five, and more preferably less than two nucleotide substitutions across each stretch of approximately 100 nucleotides. Accordingly, a nucleic acid-based assay directed to this region of the genome and an antibody-based assay directed to this region of the expressed genome, are broadly useful to detect FMDV infections across a plurality of serotypes. Preferably the assay can detect greater than two or three different serotypes, and more preferably greater than four or five different serotypes. Even more preferably, the assay can detect greater than six serotypes or all known serotypes of FMDV.

According to this method, the FMDV 3D gene can be employed as a means of detecting foot and mouth disease in susceptible populations. According to such methods, the FMDV 3D gene is identified in a biological sample obtained from a susceptible animal. Commercially important farm animals include, but are not limited to, cattle, sheep, goats, pigs, and camels. Samples can be obtained from most any tissue or cells, but are preferably obtained from vesicular fluids. Methods of the invention are preferably capable of detecting a plurality of serotypes of FMDV in animals by the detection of conserved sequences in the genome corresponding to the 3D gene. A preferred means of detection is polymerase chain reaction or PCR, most preferably RT-PCR or probe hydrolysis RT-PCR.

The nucleic acid sequence spanned by PCR primers (i.e. amplicon) is at least 50 nucleotides, preferably at least 75 nucleotides, more preferably at least 100 nucleotides, and even more preferably at least 150 nucleotides. In a particularly preferred embodiment, the genomic sequence amplified corresponds to the conserved region spanned by the primers identified in Table 2.

The general method comprises nucleic acid probes that contain sequences which, upon amplification, amplify the conserved region of the viral genome. Target sequences in the sample to be tested are amplified to increase the sensitivity of detection. Nucleic acids can be amplified directly in samples, for example using in situ PCR or immuno-PCR or RT-PCR apmplification which utilizes nucleic acid fragments coupled to pathogen-specific antibodies to increase detection sensitivity. Alternatively, nucleic acids can be analyzed after purification using, for example, DNA or RNA polymerases, PCR or another amplification technique. One of the most useful amplification techniques is PCR amplification which typically amplifies a target sequence over one million fold. Target sequences may be DNA, RNA and potentially PNA (materials with a polyamide backbone, see P. E. Nielsen et al., Science 254:1497-1500, 1991). PCR analysis of RNA, or RT-PCR, involves reverse transcription of RNA, such as mRNA sequences, into cDNA copies. These target cDNA sequences are hybridized to primers which amplify the specific sequences desired using PCR amplification. Alternatively, genomic target sequences can be amplified directly by PCR.

PCR amplifies a specific segment of DNA, the target sequence. To the segment are hybridized short oligonucleotide primers that flank the target sequence to be amplified. Primers are typically less than 35 nucleotides in length, preferably between about 8 to 25 nucleotides, or more preferably between about 12-20 nucleotides. Primers that are too short or too long may non-specifically hybridize to nucleic acid and increase background signals or reduce detection sensitivity.

Preferably the sequences of the primers are known for the primers to specifically hybridize to a relatively unique portion of nucleic acid and generate an identifiable fragment on PCR amplification. Fragments are identifiable and can be distinguished from non-specific and undesired amplification products by size. Fragments product sizes are preferably small and less than about 500 nucleotides in length, more preferably less than about 250 nucleotides and still more preferably less than 1000 nucleotides. Primers for PCR or RT-PCR detection of foot and mouth disease in susceptible animals preferably comprise a forward primer comprising the sequence 5′-ACT GGG TTT TAC AAA CCT GTG A (SEQ ID NO 2); and a reverse primer comprising the sequence 5′-GCG AGT CCT GCC ACG GA (SEQ ID NO 7). Probes used for probe hydrolysis RT-PCR for the detection of foot and mouth disease in susceptible animals preferably comprise 5′-TCC TTT GCA CGC CGT GGG AC (SEQ ID NO 5), wherein said probe is labeled with a 5′-reporter dye such as, for example, 6-carboxyfluorescein and a 3 ′-quencher such as, for example, tetramethylrhodamine.

Preferably primer pairs also do not contain complementary sequences, sequences which create intra-strand secondary structures, or complementary 3′ termini. Such structures would promote formation of artifacts and primer-dimer complexes. Extensions may be added to the 5′ termini of a primer to permit post-amplification manipulations of the PCR product without significantly affecting the amplification reaction. These 5′ extensions may be restriction enzyme recognition sites, promoter or enhancer sequences, or transcription or translation controlling signals. Primer GC content is preferably between about 40% to about 60% and long stretches of any one base should be avoided. Thermostable polymerases for PCR amplification are commercially available such as Taq DNA polymerase and AmpliTaq DNA polymerase.

Although PCR is a reliable method for amplification of target sequences, a number of other techniques can be used such as ligase chain reaction (LCR), self sustained sequence replicatin (3 SR), polymerase chain reaction linked ligase chain reaction (pLCR), gaped ligase chain reaction (gLCR), and ligase chain detection (LCD).

Amplified sequences can be detected by a variety of techniques. For example, sequences may be electrophoresed into a matrix such as, for example, an acrylamide or agarose gel, and stained with a nucleic acid stain such as ethidium bromide or silver. Alternatively, sequences can be transferred to a solid support, such as a membrane, and subsequently stained. An additional method is to label primers or chain elongating nucleotides with radioactive, fluorescent or luminescent moieties before or during amplification. Amplification products can be visualized by photographic emulsion or by scanners comprising detectors sensitive to the particular emission.

Methods of the invention can be performed rapidly on a portable instrument such as a TaqMan® assay. The present invention and methodology has many advantages over known systems including the fact that the present assay is capable of being conducted in a single tube method; it is typically capable of detecting a plurality of FMDV serotypes; and the present invention has been optimized for use on a portable instrument using dried RT-PCR reagents such as sold by Amersham Pharmacia Biotech as “Ready to Go PCR Beads.” The dried reagents can be freeze dried according to such methods of Klatser et al., J. Clin. Microbiology, vol. 36, no. 6, June 1999 pp. 1798-1800, or can be lyophilized according to known methods without any loss of potency or usage. Utilizing dried reagents, inter alia eliminates the need for cold storage during transportation, which facilitates the mobility of the assay. However, wet reagents could also be substituted if desired.

According to the present invention, a single-tube method can be used to detect and amplify RNA extracted from infected cell cultures of at least three, or up to seven or more viral serotypes, but not amplify viral RNA from viruses that cause vesicular diseases that are clinically indistinguishable from FMDV, including SVDV, VSV, and VESV. Several signals (O-BFS, Asia-1, SAT-1 and SAT-3) are amplified so strongly that if RT-PCR is employed, the fluorescence detector can even became saturated, producing a railing effect (positive, but non-sigmoidal shaped curve). By employing RT-PCR or any other rapid assay desired, viral RNA can be detected in plasma and in samples obtained from the mouth, nose, and oropharynx of animals infected with FMDV. Importantly, viral RNA can be detected in oral and nasal samples 24 to 96 hours before the onset of clinical signs. According to the present invention, it is possible to obtain detection methods and kits and products that are as sensitive, or even more sensitive than the presently accepted standard methods of virus isolation.

Detection Methods Based on Antibodies/Antigens

A second embodiment of the present invention is directed toward antibody based detection methods and assays for detecting Foot and Mouth disease in susceptible animals comprising detecting a plurality of serotypes of (FMD) virus using immuno-assay techniques utilizing antibodies to the conserved 3D protein by using the Foot-and Mouth Disease Virus (FMDV) 3D protein. The primary antibodies used recognize antigenic determinants are preferably displayed by the 3D protein. The antigenic determinant preferably comprises the products of the nucleotide sequences spanned by the primers in Table 2. Antibodies used according to this embodiment of the present invention can comprise polyclonal antibodies or monoclonal antibodies. The method can comprise antibodies or antibody fragments, preferably Fv fragments, selected from the group consisting of classes IgG, IgM, IgA, IgD and IgE, which may be derived from most any mammal, such as, for example, humans, mice, rats, goats, or any suitable species, and combinations or fragments thereof. The compositions employed in the methods may also be preserved over long periods of time by dialysis or lyophilization of the proteins to remove liquid. Lyophilized antibodies may be stable at room temperature for years. Samples tested with the present detection method can be biological tissues or antigens naturally, recombinantly or synthetically isolated.

Such methodology for protein/antibody based detection methods is conducted according to known techniques for creating an antibody/antigen. For example, one would purify a FMDV 3D protein, inject it into a suitable carrier such as a mouse or rabbit, bleed the carrier and extract the antibodies from the collected blood to create a primary antigen that could be used in connection with a kit of the present invention. Subjects could then be screened according to known techniques such as ELISA, or any other sensitive technique for detecting and measuring antigens or antibodies in a solution. For example, the solution is run over a surface to which immobilized antibodies specific to the substance have been attached. If the substance is present, antibodies will bind and their presence verified and visualized with an application of antibodies that have been tagged or labeled. In addition, the antigen can optionally be conjugated, for example, to horseradish peroxidase or another peroxidase. A substrate can be applied that turns color when it is oxidized by the peroxidase in order to confirm presence of the antigen raised by the primary antibodies. ELISA test kits would include known materials including, for example, a container having a solid phase coated with one of the subject peptide compositions, a negative control sample, a positive control sample, a specimen diluent and antibodies to species specific IGg or recombinant protein 3D. Suitable methods for immunology techniques are well known in the art and are readily from manuals and texts such as Harlow et al., (1988) Antibodies: A Laboratory Manual, Cold Spring Harbor Laboratory Press, Cold Spring Harbor, N.Y. 726 pages. Preferred techniques, as mentioned above, include ELISA as well as agglutination.

Alternatively, FMDV can be detected by screening samples to locate the 3D protein, and then comparing a targeted section of residues in the 3D gene with a standard saved as a control, and if the match between the targeted section and the standard is over 75%, then a user could conclude with a high degree of certainty is FMDV is present in the sample being tested, in other embodiments, the degree of match could be 80% or 90% or even 99% or greater, depending on the degree of certainty desired by the user.

Diagnostic Kits

Another embodiment of the invention is directed to kits that comprise DNA based diagnostic assays, such as PCR assays, which amplify the conserved region of the 3D gene. Such kits include reagents (wet or dry) that can be utilized with samples obtained from a subject to be tested for FMDV. Such reagents are well known in the art and include PCR amplication reagents such as themostable DA polymerase, deoxyologonucleotides include dATP, dGTP, dCTP and dTTP. The deoxyoligonucleotides or the primers may be labeled with a moiety to facilitate detection. Suitable labels include radionuclides, fluorescent, chemilumininscent or luminescent moieties, or coupling agents such as streptavidin, biotin or avidin, or derivatives, modification or combinations thereof. Kits also contain one or more primers that can be used for PCR amplification of the 3D gene.

In addition, the present invention provides diagnostic kits for detection of FMDV in a subject. Such kits can be used to test blood, urine, bile, cerebrospinal fluid, lymph fluid, amniotic fluid or peritoneal fluid. The kits contain antibodies that can be whole antibodies such as IgG or an antibody fragment such as an Fab fragment. Kits according to the present invention are used in connection with an antibody library that may be labeled with a detectable label or the kit may further comprise a labeled secondary antibody that recognizes and binds to antigen-antibody complexes. Preferably, the detectable label is visually detectable such as an enzyme, fluorescent chemical, luminescent chemical or chromatic chemical, which would facilitate determination of test results for the user. Diagnostic kits may further include agents to increase stability, shelf-life inhibit or prevent product contamination and increase detection speed. Useful stabilizing agents include water, saline, alcohol, glycols including polyethylene glycol, oil, polysaccharides, salts, glycerol, stabilizers, emulsifiers and combinations thereof. Antibacterial agents and agents to optimize speed of detection may also be employed if desired for any reason.

The assay is specific for FMDV, sensitive, rapid and easy to perform. Further, the assay can be performed in the field. Accordingly, the total time to obtain assay results is less than four hours, preferably less than two hours, and more preferably less than one hour. The ability to detect viral RNA in the saliva of sheep, pigs and cattle makes this assay a valuable tool for the monitoring and early diagnosis of FMDV in rural farm areas and help in controlling the spread of the disease. The present invention would also be useful in the aftermath of an epizootic to ensure that the disease had truly been stamped out.

There can be in some instances up to 100% concordance between positive results of virus isolation and the RT-PCR assay when performed on nasal, oral, oral pharyngeal, epithelial tissue, and plasma samples from experimentally infected and control animals. Imine-inactivated samples can be amplified with little loss of signal, indicating that noninfectious RNA can still be detected. This may be of particular importance when evaluating suboptimal clinical samples in which viral infectivity has been lost.

In addition to being sensitive and specific for FMDV, tests, methods and kits according to the present invention are rapid, easy to perform, and portable, thus ensure rapid disease diagnosis. Total turnaround time for results can be approximately two hours, including RNA extraction. The fact that the present invention is capable of detecting viral RNA directly from vesicular fluid is especially important, because this could potentially make the process faster (45 minutes) and logistically much more manageable in the field, because the assays can be performed without the need for refrigeration or centrifugation.

A diagnostic test for FMDV according to the present invention has several advantages, compared with traditional diagnostic methods. As a single-tube RT-PCR test that uses dried reagents, tests can be produced in lots, which undergo quality control testing before distribution, making test results more reproducible. Sample testing only involves rehydration of the tube and addition of the sample, which makes the assay highly standardized and less prone to variation, cross-contamination, and operator error. Preferably, the RT-PCR is run for 45 cycles; however, a run of 55 cycles demonstrates the full shape of the amplification curves and their robustness after 45 cycles in the dried test. In addition, while PCR (preferably RT-PCR) is a preferred method for comparing the targeted probes and primers of the present invention, other techniques may be employed to achieve the same end result, that being obtaining a measurement of the degree of matching between a base pairs in a particular targeted region in the 3D gene with the same area in the 3D gene in a sample.

The assay can be used in a variety of field contexts in combination with appropriate bio-safety measures to quickly identify FMDV-infected herds and define infected and disease-free zones, almost in real-time. The assay is also useful in the aftermath of an epizootic to identify carrier animals, distinguish infected from vaccinated animals, and screen out closely related viral infections.

The following examples are offered to illustrate embodiments of the present invention, but should not be viewed as limiting the scope of the invention.

### EXAMPLES

### Example 1

Specific oligonucleotide primers and a fluorogenic probe were designed to target a highly conserved region within the FMDV 3D gene. The RT-PCR assay was a rapid single-tube method consisting of a 10 minute RT step, linked to a 45 cycle PCR at 95° C. and 60° C. that generated a fluorogenic signal in positive samples. Primer and probe sequences are listed in Table 1. The assay was optimized for use on the SmartCycler™ (Cepheid, Inc., Sunnyvale, Calif.), a 27-pound portable instrument that can be operated by a lap-top computer.

At the Plum Island Animal Disease Center, NY, USA, epithelial tissue and saliva samples were collected seven days post infection from cows, sheep and pigs that had been experimentally infected with an FMDV of Type O as part of the Foreign Animal Disease (FAD) course at Plum Island. Blood samples were also obtained 5 days following infection of pigs with a virus of type A. Viral RNA was extracted from the samples using commercially available RNA extraction kits (Qiagen, Valencia, Calif.). The initial evaluation of the assay was conducted using 2.5 μl of extracted RNA in a final reaction volume of 25 μl. The assay was further modified by dehydrating the RT-PCR reaction mixture into 100 μl volume optical reaction tubes that could be stored and transported at ambient temperatures. The dried reaction tubes were then rehydrated for testing with 40 μl of diluent and up to 60 μl of test material. This modification of the assay allowed for a 25× increase in sample volume size, which greatly increased the sensitivity and performance of the assay.

These results demonstrate that the present assay has a specificity of one hundred percent when testing RNA extracted from viruses representing the seven FMDV serotypes: A, O, C, Asia-1, Sat 1, Sat 2 and Sat 3, but does not amplify the RNA of the viruses of swine vesicular disease, vesicular stomatitis, and vesicular exanthema of swine, agents causing diseases which are clinically indistinguishable from FMD. There was one hundred percent concordance between positive viral cultures and the RT-PCR assay when testing epithelial tissue, saliva, and whole blood samples from experimentally infected and control animals. Additionally, one sample which was negative in culture was positive by RT-PCR, and four samples with imine inactivated virus (used for vaccine studies) could be amplified with very little loss of signal when compared to untreated. samples, indicating that even though the inactivated virus failed to replicate in culture, the RNA is still detected. The dynamic range of the assay is at least 6 log dilutions for both O and Asia-1 serotypes. The estimated sensitivity of 10 copies of viral RNA per volume tested was obtained by observing the last log dilution of FMDV (of known TCID₅₀ concentration) giving a positive result by RT-PCR, adjusted for the volume used in RNA extraction and the volume tested.

It was also determined whether virus could be detected in tissues or nasal and mucosal swabs of infected animals before disease signs were manifested. A steer which had been infected by inoculation of the tongue with a virus of serotype O, antigenically similar to that causing the outbreak in the UK, was placed in contact with four susceptible steers. Although the contact steers did not show signs of disease at 48 hours post contact, viral RNA was clearly detected by our technique in oral, nasal, probing, and plasma samples.

### Example 2

According to Example 2, an assay was designed taking into consideration several FMDV strains and other closely related genera as near neighbors. Table 3 includes the FMVD serotypes detected by the FMDV assay.

TABLE 3
FMDV Specificity Panel

| Virus | Strain or Source | Material |
|---|---|---|
| FMDV-A | Plum Island | RNA |
| FMDV-O | Plum Island | RNA |
| FMDV-C | Plum Island | RNA |
| FMDV-SAT 1 | Plum Island | RNA |
| FMDV-SAT 2 | Plum Island | RNA |
| FMDV-SAT 3 | Plum Island | RNA |
| FMDV-ASIA 1 | Plum Island | RNA |

Based on available phylogenetic studies, near neighbors were identified and are listed in Table 4. Initial sensitivity and specificity experiments will be conducted on the serotypes listed in Table 3 and the near neighbors listed in Table 4.

TABLE 4
Viral specificity Panel (All Flaviviruses)

| Viral near neighbors | Strain or Source |
|---|---|
| Dengue 2 | New Guinae C |
| St. Louis Encephalitis | TBH-28 |
| Japanese Encephalitis | SA 14-14-2 |
| West Nile-NY Crow | 394-99, ITRI |
| Kunjin | K5374, ITRI |
| Murray Valley Encephalitis | IITRI |
| Yellow Fever | 17D-213, InDx Inc. |

Table 5 is a general specificity panel that has been used for specificity testing on previous assays.

TABLE 5
General Specificity Panel Results

| Organism | Material |
|---|---|
| B. cepaciae | DNA, EPA Strain G4 |
| B. cepaciae | DNA, EPA Strain G4 |
| B. cereus | DNA, ATCC 11778 |
| B. sphaericus | DNA, ATCC 4525 |
| C. freundii | DNA, BMI |
| C. sporogenes | DNA, ATCC 1955 |
| E. faecalis | DNA, ATCC 13048 |
| F. philomoragia | DNA, ATCC 25015 |
| L. acidophilus | DNA, ATCC 9857 |
| Human DNA | DNA, (Sigma D-7011) |
| P. acnes | DNA, ATCC 6919 |
| P. aeruginosa | DNA, ATCC 10145 |
| P. anaerobius | DNA, ATCC 27337 |
| P. putida | DNA, ATCC 12633 |
| S. cerevisiae | DNA, ATCC 2366 |
| S. epidermis | DNA, ATCC 14990 |
| S. mutans | DNA, ATCC 25175 |
| V. parahaemolyticus | DNA, ATCC 17802 |
| Y. pseudotuberculosis | DNA, ATCC 29833 |
| S. cervisiae | DNA, ATCC 2366 |

Table 6 is a panel of DNA extracts from a number of cell lines that are routinely used for viral isolation.

TABLE 6
Cell Line Extracts for Viral Specificity Testing

| Cell Line | Source | Extraction Date | Material |
|---|---|---|---|
| BHK-21 | ITRI | 3/16/00 | DNA |
| C6/36 | ITRI | 4/14/00 | DNA |
| HeLa | ITRI | 3/31/00 | DNA |
| L929 | ITRI | 4/14/00 | DNA |
| Sf-9 | ITRI | 4/5/00 | DNA |
| SL-29 | ITRI | 4/24/00 | DNA |
| Vero76 | ITRI | 3/31/00 | DNA |

The viral samples were diluted 10-fold (log dilutions) in 1×TE to establish the threshold of probable detection (TPD) and the limit of detection (LOD). Further specificity experiments were conducted on samples from Plum Island. At Plum Island, the assay tested a panel of isolates from their extensive collection. The strain sequences used in the design of this assay are listed in Table 4. Sensitivity and specificity measurements conducted using the assay to test the viral stocks listed in Table 3. Threshold of probable detection (TPD) and limit of detection (LOD) measurements were made utilizing dilutions of viral stock. The LOD was determined by the lowest level at which detection is periodically observed. The TPD was established by determining the lowest dilution at which at least 19 out of 20 reactions are positive.

### Example 3

FMDV isolates that represented all 7 serotypes (A-12: O-BFS; O-South Korea: C3 Resende: Asia 1 PAK 1/54: SAT 1: SAT 2: and SAT 3 were grown in monolayers of a continuous bovine kidney cell line (LF-BK). Viral infectivity was measured in 96-well plates by use of standard methods. Viruses that cause similar clinical signs, namely, swine vesicular disease virus (SVDV)-UK and It-1/66, vesicular stomatitis virus (VSV)-Indiana, and vesicular exanthema of swine virus (VESV) A-48, were grown in monolayers of pig kidney cells (IBRS2), baby hamster kidney cells (BHK-21), and LF-BK cells.

Viral RNA was extracted from cell culture supernatants or plasma, whole blood, or tissue in a class II biosafety cabinet (blower left on permanently) by use of commercially available kits following manufacturers' instructions. To minimize the potential for contamination, RNA extraction and RT-PCR testing were performed in separate laboratories that used aerosol barrier tips for all steps of each procedure.

Archived clinical samples from a foreign animal disease course held at Plum Island Animal Disease Center, which included epithelial tissue and saliva samples obtained from steers, sheep, and pigs seven days after contact exposure to a pig that had been infected by ID inoculation of 10⁵ tissue culture infective doses (TCID)₅₀ of a FMDV (type O, Brugge), were examined. Additional clinical samples included blood samples obtained from pigs five days after ID inoculation of 10⁵ TCID₅₀ of a type A virus. The blood samples were divided into two aliquots, one of which was inactivated with the imine compound acetyl ethyleneimine, as described, and the other of which was untreated. Both aliquots underwent RNA extraction and were tested in parallel for viral infectivity and viral RNA.

In a controlled study, a group of experimentally infected animals were evaluated. Prior to exposure of the animals to FMDV, control samples were collected and included plasma, oral and nasal swab specimens, and oral-pharyngeal fluids (60 samples from 5 cattle, 5 swine, and 5 sheep). One steer, one pig, and one sheep were infected by ID inoculation of 10⁷ TCID₅₀ of a serotype O virus from South Korea that was antigenically similar to the type O-Pan Asian strain that caused the outbreak in the United Kingdom. Twenty four hours later the animals were placed in separate rooms that contained four FMDV-free, healthy animals of the same species. Oral and nasal swab specimens, oral-pharyngeal fluid specimens, and blood samples were obtained at 0 (before exposure), 4, 8, 12, 16, 24, 48, 72, 96, 120, and 144 hours, and the animals were observed for the onset of fever and clinical signs of FMDV. The oral swab specimens were obtained by swabbing under the tongue and an area of contact between the lower gum and the inner surface of the lower lip. Samples were collected by use of dedicated sterile cup probangs, or individual sterile tubes that contained cotton-tipped wood applicators in which the shaft was broken against the side of the vial and the tube capped. Operators changed sterile latex gloves after collecting samples from each of the animals. Samples were assayed for infectious virus and viral RNA. Additionally, oral swab specimens from a cohort of uninfected cattle (n=241), which included 110 cows, 100 calves, and 31 sick calves (respiratory illness of unknown etiology) were obtained from the Meat Animal Research Center, Agricultural Research Service, USDA, Clay Center, Nebr. and tested for FMDV RNA. All tests were performed in a masked fashion in parallel with viral isolation.

Real-time RT-PCR Assay

The FMDV nucleotide sequences were retrieved from GenBank and aligned by use of sequence alignment software. Specific oligonucleotide primers and a fluorogenic probe were designed to target a highly conserved region within the FMDV RNA polymerase (3D) gene sequence alignment. The location and sequence of the primers and probes were as follows: forward primer starting with base position 6769 (GenBank AF189157) 5′-ACT GGG TTT TAC AAA CCT GTG A (SEQ ID NO 2); reverse primer, base 6875, 5′-GCG AGT CCT GCC ACG GA (SEQ ID NO 7), and probe, base 6820, 5′-TCC TTT GCA CGC CGT GGG AC (SEQ ID NO 5). The probe was labeled with a 5′-reporter dye, 6-carboxyfluorescein and a 3′-quencher, tetramethylrhodamine. A blast search analysis of the primer and probe sequences confirmed one hundred percent homology with five of the seven serotypes of FMDV, with the exception of a single base mismatch located within the forward primer binding area of SAT 2. Sequence information for the 3D genomic region of serotypes SAT 1 and SAT 3 is presently not available.

The assay was designed as a single-tube reverse transcriptase polymerase chain reaction (RT-PCR) probe hydrolysis assay. Reagents were used to prepare master-mix recipes according to the manufacturer's guidelines for individual component concentrations. Final PCR reactions for a 25-μl volume using 2.5 μl of template were performed with the 5× buffer solution supplied by the kit and the addition of Mn(OAc)₂ (5 mM), primers (0.3 μM), probe (0.3 μM, dATP/CTP/GTP (0.1 mM), dUTP (0.2 mM), rTth DNA polymerase (0.1 U/μl), bovine serum albumin (0.1 μg/μl), and trehalose (0.5M). The RT-PCR reaction mixture was dried within the reaction tubes. For sample testing, dried reagents were rehydrated with sample and diluent and run with cycling conditions that consisted of a 10-minute RT step at 60° C., linked to a 45-cycle PCR (95° C. for 2 seconds and 60° C. for 30 seconds), which generated a fluorogenic signal in samples with positive results. The assay was optimized for use on a portable 22-pound real-time thermocycler that can be operated by a lap-top computer. Initial development and evaluation of the assay was performed by use of cell culture-derived virus and included a comparison of the standard wet assay run in parallel with the vitrified dry assay to ensure that there was no loss in sensitivity associated with the drying process. Positive and negative controls consisting of viral RNA extracted from cell culture supernatant and a no-template control were included with each RT-PCR run.

Analysis of isolates that represented the seven FMDV serotypes had infectivity titers of approximately 1×10⁸ TCID₅₀/100 μl when grown in monolayers of BHK-21 cells. Viral RNA extracted from these stock viruses was tested by use of RT-PCR and resulted in positive (cycle) threshold values ranging from 17 to 20 PCR cycles, demonstrating robust amplification signals for each of the samples that represented all seven serotypes, including SAT 1 and SAT 3, for which sequence information for the 3D region was not available.

To assess the specificity of the RT-PCR, tissue culture supernatants of other viruses that cause vesicular diseases including SVDV (UK and It-1/66), VSV (Indiana), and VESV, each with a titer of approximately 10⁸ to 10⁹ TCID₅₀/100 μl in LF-BK cells, were tested. In no instance was a positive amplification signal obtained for any molecular target other than FMDV RNA, which indicated a specificity of one hundred percent for the selected panel. Results of the assay were also negative when tested for cross reactivity against a panel of non-related RNA viruses available in our laboratory, including dengue, yellow fever, West Nile, and other flaviviruses, each with a titer of approximately 10⁵ to 10⁶ plaque-forming units (PFU)/ml, and against DNA extracts from bovine blood, pig macrophages, and cell lines that are commonly used for viral isolation (BHK-21, C6/36, HeLa, L929, SF-29, SL-29, and Vero-76). To further demonstrate the specificity of our FMDV assay, a similar RT-PCR test specific for SVDV gave positive results for SVDV, but did not amplify FMDV type A-12. Results obtained from testing 15 animals before exposure (n=60 samples) and 241 cohort animals were negative, which indicated that there were no false positives when animals that were not infected with FMDV were tested. These results indicated an assay specificity of one hundred percent for testing healthy control animals.

To assess assay sensitivity, viral RNA was extracted from supernatants of FMDV SAT 2 and British Field Strain type O (O-BFS) infected BHK-21 cells. Viral RNA was diluted in log-10 steps in 1×Tris-EDTA and tested to determine the end point dilution at which a positive amplification signal could be obtained. The amplification signals indicated that with use of the test in either the standard (wet) or vitrified (dried) format, viral RNA was still detected and amplified after a 10⁸ dilution of starting material, which had titer of 10⁸ PFU/100 μl. Results of spectroscopic and electron microscopic studies indicated that the particle-to-infectivity ratio for FMDV was approximately 1,000:1¹²; thus, 10⁹ PFU/ml may represent as many as 10¹² RNA copies. An amplification curve representing a 10⁸ dilution is then equal to a detection limit of 10⁴ RNA copies/ml. Adjusting for the sample volume used for extraction (140 μl), the yield of purified RNA (80 μl), and the volume tested (2.5 μl), the estimate of assay sensitivity was between 10 and 100 virus genomes/volume tested. It must be emphasized that this was an estimate because the efficiencies of the RNA extraction, the reverse transcription step, and the PCR cycling itself are less than one hundred percent efficient and the physical-particle-to-infectious-particle ratio was also an estimate.

In addition, a variety of oral, nasal, oral-pharyngeal, and plasma samples from cattle, pigs, and sheep. In the first animal experiments in which steers, pigs and sheep were infected with virus of serotype O, Brugge there was one hundred percent concordance between positive results of viral cultures and the RT-PCR assay when testing epithelial tissue, saliva, and blood samples from experimentally infected and control animals. Additionally, one sample with negative results in culture (saliva, sheep one) had positive results by use of RT-PCR assay, and 5 imine-inactivated samples could be amplified with no loss of signal, compared with untreated samples, judged by the number of cycles required to reach the same level of amplification.

In a second experiment, cattle, pigs, and sheep were infected by contact with an animal from the same species that had been infected with a virus of serotype O, isolated in South Korea, which is similar to the virus presently causing the disease in the United Kingdom. Of the four naïve cattle that were placed in contact with an experimentally infected steer, two developed vesicular lesions within 48 hours, one at 96 hours, and the fourth at 144 hours. However, all four contact animals had positive results of RT-PCR assay and viral culture by 72 hours with all sample types. The assay detected FMDV 24 to 96 hours prior to the appearance of clinical signs in all animals. Viral RNA was detected in all the contact steers and all sample types (except oral swabs), at least 24 hours before viral culture became positive.

Of the four naïve pigs placed in contact with an infected pig, none developed fever, but all four developed clinical lesions typical of FMD within 96 hours after contact. For oral samples obtained from one contact pig, results of the RT-PCR assay and viral culture were positive at least 24 hours before clinical signs were evident. These data are typical of the other two pigs that had positive results by RT-PCR assay and viral culture. Positive results were obtained from all samples types in the three pigs except for plasma samples, which were positive by RT-PCR only and not viral culture at 122 or 144 hours. The fourth pig had negative results by RT-PCR assay and viral culture in samples collected up to 72 hours after contact, but subsequently developed lesions and neutralizing antibodies after sampling was concluded.

Of the four naïve sheep placed in contact with an infected sheep, only one developed fever. This sheep also had positive results by RT-PCR assay and viral culture for oral and oral-pharyngeal samples, but had negative results by use of both methods for nasal swab samples. A second sheep also had positive results by RT-PCR assay and viral culture with oral swab and oropharyngeal samples. Of the remaining two sheep, virus were undetectable either by use of RT-PCR assay or culture in samples collected up to 120 hours after contact. However, one of these sheep had neutralizing antibodies at 25 days after contact exposure, which indicated that the sheep did actually become infected sometime after the sampling period was concluded (120 hours after exposure), or alternatively, had a subclinical infection that was not detected by use of our assay or by use of virus isolation. One sheep clearly did not become infected, as indicated by negative serology results (no neutralizing antibodies at 25 days after contact exposure).

Just over one hour of the two-hour testing process was devoted to preparation of the samples, which involves extraction of viral RNA. It was found, however, that it was possible to amplify viral RNA directly from vesicular fluid from an infected animal without prior extraction of RNA, thus halving the time necessary for the analysis.

The experiments of example 3 were conducted with reagents that had been dried into the test cuvette devices by use of trehalose, which stabilizes the reaction mixture and allows the sample to be stored and transported at room temperature (such as 20 to 22° C.). Results of accelerated stability studies modeled after the Food and Drug Administration guidelines used for the manufacture of drugs and drug products indicate a minimum two-year shelf life for the test at ambient temperature. It was found that the vitrification process actually improved the performance of the assay. Not only did the process increase the sensitivity of the test by at least 10-fold, but the shape of the curves at the low end of the sensitivity range was improved from a flat shape to a more sigmoidal shape. By comparison of the amplification signals of an RNA dilution series, it was clear that the slope of the curves was superior, and the 10¹⁰ dilution represented a 100-fold increase in sensitivity by use of the vitrified reagents versus the standard wet reagents.

Additionally, only one sample had negative results of virus isolation at 72 hours had positive results by use of RT-PCR assay, whereas subsequent samples collected at 96 hours had positive results with both assay methods. Notably, of the 301 samples with negative results, there were no false positive RT-PCR reactions.

After steer, pig, and sheep were infected with serotype O FMDV, twenty-four hours later, animals were placed in separate rooms that contained four FMDV-free, healthy animals of the same species. Oral and nasal swab specimens, oral-pharyngeal specimens, and blood samples were obtained at frequent intervals and animals were observed for fever and clinical signs of FMD. Samples were assayed for infectious virus and viral RNA.

The assay detected viral RNA representing all seven FMDV serotypes grown in tissue culture, but did not amplify a panel of selected viruses that included those that cause vesicular diseases similar to FMD; thus the assay had a specificity of one hundred percent, depending on the panel selected. The assay also met or exceeded sensitivity of viral culture on samples from experimentally infected animals. In many instances, the assay detected viral RNA in the mouth and nose 24 to 96 hours before the onset of clinical disease.

Other embodiments and uses of the invention will be apparent to those skilled in the art from consideration of the specification and practice of the invention disclosed herein. Additional advantages, features and modifications will readily occur to those skilled in the art. Therefore, the invention in its broader aspects is not limited to the specific details, and representative devices, shown and described herein. Accordingly, various modifications may be made without departing from the spirit or scope of the general inventive concept as defined bye the appended claims and their equivalents.

All references cited herein, including all U.S. and foreign patents and patent applications and specifically U.S. provisional application No. 60/291,636, filed May 18, 2001, are specifically and entirely hereby incorporated herein by reference. It is intended that the specification and examples be considered exemplary only, with the true scope and spirit of the invention indicated by the following claims.

1. An isolated nucleic acid comprising a sequence selected from the group consisting of any of at least ten contiguous nucleotides:
of a portion of the 3D coding region of a FMDV genome, wherein said portion comprises the 3′-terminal third of said coding region or of the complement of said portion; or
that hybridizes under stringent hybridization conditions to said portion or the complement of said portion.

2. The nucleic acid of claim 1 wherein the at least ten contiguous nucleotides comprises at least fifteen contiguous nucleotides.

3. The nucleic acid of claim 1 wherein the at least ten contiguous nucleotides comprises at least twenty contiguous nucleotides.

4. The nucleic acid of claim 1 wherein the at least ten contiguous nucleotides comprises at least thirty contiguous nucleotides.

5. The nucleic acid of claim 1 wherein the at least ten contiguous nucleotides comprises at least thirty five contiguous nucleotides.

6. The nucleic acid of claim 1 wherein the at least ten contiguous nucleotides comprises at least fifty contiguous nucleotides.

7. The nucleic acid of claim 1 wherein said nucleic acid comprises DNA or PNA.

8. The nucleic acid of claim 1 wherein the 3′-terminal portion comprises a sequence selected from the group consisting of positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype.

9. The nucleic acid of claim 1 wherein the 3′-terminal portion comprises a sequence selected from the group consisting of positions 1,102 to 1,401 of the 3D coding region of an Asia 1 serotype, and positions 1281 to 1582 of the 3D coding region of a C-1-Santa Pau (C-s8) replicase genotype.

10. The nucleic acid of claim 1 wherein the foot and mouth disease virus genome is selected from the group consisting of the genomes of serotype A, serotype C, serotype O, serotype Asia 1, serotype Sat 1, serotype Sat 2, and serotype Sat 3.

11. A pair of two different nucleic acids, each of which comprises a nucleic acid of claim 1, wherein the pair is capable of priming a PCR that amplifies a region of nucleic acid within said portion.

12. A kit for the detection of a FMDV infection in a patient comprising the pair of nucleic acids of claim 11.

13. The kit of claim 12 which is capable of detecting a plurality of serotypes of FMDV.

14. A vector comprising the nucleic acid of claim 1.

15. A cell containing the vector of claim 14.

16. The pair of two different nucleic acids wherein each nucleic acid of said pair is selected from the group consisting of SEQ ID NOS. 1-14.

17. A method for detecting a FMDV infection in a patient comprising:
amplifying a portion of nucleic acid of a biological sample obtained from said patient by PCR amplification to produce an amplification product wherein said amplification product contains a sequence derived from the 3D coding region of a FMDV genome; and
detecting said FMDV infection in said patient by the presence of said amplification product.

18. The method of claim 17 wherein the FMDV infection is caused by any of the FMDV serotypes selected from the group consisting of Asia, A, C, O, Sat 1, Sat 2, and Sat 3.

19. The method of claim 17 wherein the patient is selected from the group consisting of cattle, horses, pigs, sheep, camels, and goats.

20. The method of claim 17 wherein amplification comprises PCR RT-PCR, or probe hydrolysis RT-PCR amplification.

21. The method of claim 17 wherein the biological sample is selected from the group consisting of a sample of tissue, fluid or combination of tissue and fluid obtained from the patient.

22. The method of claim 21 wherein the sample comprises material collected from a vesicle or lesion of the patient.

23. The method of claim 17 wherein nucleic acid is isolated from the sample prior to amplification.

24. The method of claim 17 wherein the amplification product contains a sequence derived from a 3′-terminal portion of the 3D coding region of FMDV.

25. The method of claim 24 wherein the 3′-terminal portion is selected from the group consisting of positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype.

26. The method of claim 1 which can distinguish a FMDV-infected patient from a patient infected with one or more of the viruses selected from the group consisting of swine vesicular disease virus, vesicular stomatitis virus, and vesicular exanthema of swine virus.

27. The method of claim 1 which can distinguish a FMDV-vaccinated patient from a FMDV-infected patient.

28. A method for detecting an infection caused by any of a plurality of serotypes of FMDV comprising:
amplifying a portion of nucleic acid of a biological sample obtained from said patient by PCR amplification to produce an amplification product wherein said amplification product contains a sequence derived from the 3D coding region of a FMDV genome; and
detecting said FMDV infection in said patient by the presence of said amplification product.

29. The method of claim 28 wherein the plurality of serotypes of FMDV comprises at least three serotypes selected from the group consisting of serotypes Asia 1, A, C, O, Sat 1, Sat 2, and Sat 3.

30. The method of claim 28 wherein the plurality of serotypes of FMDV comprises at least four serotypes selected from the group consisting of serotypes Asia 1, A, C, O, Sat 1, Sat 2, and Sat 3.

31. The method of claim 28 wherein the plurality of serotypes of FMDV comprises at least five serotypes selected from the group consisting of serotypes Asia 1, A, C, O, Sat 1, Sat 2, and Sat 3.

32. The method of claim 28 wherein the plurality of serotypes of FMDV comprises at least six serotypes selected from the group consisting of serotypes Asia 1, A, C, O, Sat 1, Sat 2, and Sat 3.

33. The method of claim 28 wherein the plurality of serotypes of FMDV comprises serotypes Asia 1, A, C, O, Sat 1, Sat 2, and Sat 3.

34. The method of claim 28 wherein the portion of nucleic acid amplified is derived from a sequence selected from the group consisting of the sequences of positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype.

35. The method of claim 28 wherein the amplification product is formed from two different primers each of which is selected from the group consisting of SEQ ID NOS 1-14.

36. A kit for performing the method of claim 28.

37. The kit of claim 36 wherein the method can be performed and an FMDV infection detected within about 2 hours.

38. The kit of claim 36 which further contains dried reagents for RT-PCR analysis of said biological sample plus trehalose.

39. The kit of claim 36 which can be stored at room temperatures for at least one year.

40. A kit for detecting any of a plurality of serotypes of FMDV comprising a pair of primers for PCR amplification of at least a portion of a 3D coding region of a FMDV genome.

41. The kit of claim 40 wherein the plurality comprises three, four, five, six or seven different serotypes of FMDV.

42. The kit of claim 40 wherein each primer comprises a sequence selected from the group consisting of SEQ ID NOS. 1-14.

43. The kit of claim 40 wherein the 3D coding region is selected from the group consisting of positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype.

44. A method for detecting an infection of a patient caused by any of a plurality of serotypes of FMDV comprising:
contacting a biological sample obtained from said patient with an antibody directed against a 3D coding region of an expressed portion of a FMDV genome to form antibody/antigen complexes; and
detecting said FMDV infection in said patient by the presence of antibody/antigen complexes.

45. The method of claim 44 wherein the plurality of serotypes of FMDV comprises at least three serotypes selected from the group consisting of serotypes Asia 1, A, C, O, Sat 1, Sat 2, and Sat3.

46. The method of claim 44 wherein the plurality of serotypes of FMDV comprises at least four serotypes selected from the group consisting of serotypes Asia 1, A, C, O, Sat 1, Sat 2, and Sat3.

47. The method of claim 44 wherein the plurality of serotypes of FMDV comprises at least five serotypes selected from the group consisting of serotypes Asia 1, A, C, O, Sat 1, Sat 2, and Sat 3.

48. The method of claim 44 wherein the plurality of serotypes of FMDV comprises at least six serotypes selected from the group consisting of serotypes Asia 1, A, C, O, Sat 1, Sat 2, and Sat 3.

49. The method of claim 44 wherein the plurality of serotypes of FMDV comprises serotypes Asia 1, A, C, O, Sat 1, Sat 2, and Sat 3.

50. The method of claim 44 wherein the antibody is a monoclonal antibody, a polyclonal antibody, or an antibody fragment.

51. The method of claim 44 wherein the expressed portion comprises a product of the sequence of: positions 6685 to 6996 of an O serotype, isolate 01 Campos; positions 7769 to 8076 of an O serotype; positions 7401 to 7712 of an O serotype, isolate 01K; positions 7400 to 7707 of an O serotype, isolate O/SKR/2000, positions 7319 to 7626 of an O serotype, isolate Chu-Pei strain; positions 7336 to 7643 of an O serotype, isolate Tau-Yuan TW9 strain, positions 7711 to 8018 of a C serotype, strains rp146, rp99 and c-s8c1; and positions 7371 to 7678 of an Sat 2 serotype.

52. A kit for performing the method of claim 44.

53. An antibody that specifically binds to a polypeptide expressed from a 3D coding region of a FMDV genome.

54. The antibody of claim 53 which is a monoclonal antibody or a polyclonal antibody, or a fragment thereof.

55. A kit for detecting FMDV infection in a patient comprising the antibody of claim 53.

56. The kit of claim 55 wherein the antibody is labeled with a detectable label.

57. The kit of claim 56 wherein the detectable label is fluorescent.

57. The kit of claim 54 which is capable of detecting an infection caused by any of a plurality of serotypes of FMDV.

58. The kit of claim 57 wherein the plurality comprises greater than three, greater than four, greater than five, or greater than six serotypes of FMDV.
